(19) United States
(12) Patent Application Publication
(10) Pub. No.: US 2022/0142521 A1
(43) Pub. Date: May 12, 2022

(54) METHODS AND APPARATUS FOR PREDICTING WHETHER AND WHEN A HYPO/HYPER ANALYTE CONCENTRATION EVENT WILL OCCUR

(71) Applicant: Ascensia Diabetes Care Holdings AG, Basel (CH)

(72) Inventor: Anthony P. Russo, New York, NY (US)

(21) Appl. No.: 17/520,621

(22) Filed: Nov. 5, 2021

Related U.S. Application Data
(60) Provisional application No. 63/112,152, filed on Nov. 10, 2020.

(51) Int. Cl. A61B 5/145; A61B 5/00

(52) U.S. Cl. CPC A61B 5/14532; A61B 5/6801; A61B 5/7275; A61B 5/7267

(57) ABSTRACT

A method of predicting an analyte concentration trend to provide a user with an opportunity to take therapeutic measures, if needed, includes receiving a plurality of past measured analyte concentrations between a time t₀ of a most recent measured analyte concentration and a time t_{P} of an earlier measured analyte concentration; calculating a data set comprising differences in measured analyte concentrations between consecutive measured analyte concentrations between the time t_{P} and the time t₀; and predicting whether a hypo/hyper analyte concentration event will occur within a predetermined time period after the time t₀ based at least in part on the first data set. Other methods and apparatus are also disclosed.

### CROSS REFERENCE TO RELATED APPLICATION

This claims the benefit of U.S. Provisional Patent Application No. 63/112,152, filed Nov. 10, 2020, the disclosure of which is hereby incorporated by reference herein in its entirety for all purposes.

### FIELD

The present disclosure relates to apparatus and methods for continuous analyte monitoring.

### BACKGROUND

Continuous analyte monitoring (CAM), such as continuous glucose monitoring (CGM), has become a routine monitoring operation, particularly for individuals with diabetes. CAM provides real-time analyte analysis (e.g., analyte concentrations) of an individual's body fluid. In the case of CGM, real-time glucose concentrations of an individual's interstitial fluid are provided. By providing real-time glucose concentrations, therapeutic and/or clinical actions may be timelier applied to individuals being monitored, thus better controlling glycemic conditions.

Improved CAM and CGM methods and apparatus are desired.

### SUMMARY

In some embodiments, a method of predicting an analyte concentration trend is provided. The method includes the following: receiving a plurality of past measured analyte concentrations between a time t₀ of a most recent measured analyte concentration and a time t_{P} of an earlier measured analyte concentration; calculating a first data set comprising differences in measured analyte concentrations between consecutive measured analyte concentrations between the time t_{P} and the time t₀; and predicting whether a hypo/hyper analyte concentration event will occur within a predetermined time period after the time t₀ based at least in part on the first data set.

In some embodiments, a method of predicting a glucose concentration trend is provided. The method includes the following: receiving a plurality of past measured glucose concentrations between a time t₀ of a most recent measured glucose concentration and a time t_{P} of an earlier measured glucose concentration; calculating a first data set comprising differences in measured glucose concentrations between consecutive measured glucose concentrations between the time t_{P} and the time t₀; calculating a second data set comprising differences in measured glucose concentrations between a measured glucose concentration at the time t₀ and each measured glucose concentration before the time t₀; and predicting whether a hypo/hyper glycemic event will occur within a predetermined time period after the time t₀ based at least in part on the first data set and the second data set.

In some embodiments, an event detector is provided. The event detector includes a processor configured to execute computer-readable instructions that cause the processor to: receive a plurality of past measured glucose concentrations between a time t₀ of a most recent measured glucose concentration and a time t_{P} of an earlier measured glucose concentration; calculate a first data set comprising differences in measured glucose concentrations between consecutive measured glucose concentrations between the time t_{P} and the time t₀; and predict whether a hypo/hyper glycemic event will occur within a predetermined time period after the time t₀ based at least in part on the first data set.

Other features, aspects, and advantages of embodiments in accordance with the present disclosure will become more fully apparent from the following detailed description, the claims, and the accompanying drawings that describe, define, and illustrate a number of example embodiments and implementations. Various embodiments in accordance with the present disclosure may also be capable of other and different applications, and its several details may be modified in various respects, all without departing from the scope of the claims and their equivalents. Accordingly, the drawings and descriptions are to be regarded as illustrative in nature, and not as restrictive.

### BRIEF DESCRIPTION OF THE DRAWINGS

The drawings, described below, are for illustrative purposes only and are not necessarily drawn to scale. The drawings are not intended to limit the scope of the disclosure in any way. Like numerals are used throughout to denote the same or like elements.

FIG. 1 illustrates a block diagram of a continuous glucose monitoring system including a wearable device and an external device in accordance with embodiments described herein.

FIG. 2A illustrates a graph showing an example of measured glucose concentrations including a hypoglycemic event of a user in accordance with embodiments described herein.

FIG. 2B illustrates a graph showing an example of measured glucose concentrations including a hyperglycemic event of a user in accordance with embodiments described herein.

FIG. 3A illustrates a flowchart of a first method of predicting whether glucose concentrations will cross a threshold in accordance with embodiments described herein.

FIG. 3B illustrates a flowchart of a second method of predicting whether glucose concentrations will cross a threshold in accordance with embodiments described herein.

FIG. 4 illustrates a block diagram showing glucose concentration calculations that may be received by an event detector to predict a glucose concentration trend or behavior in accordance with embodiments described herein.

FIG. 5 illustrates an embodiment of an event detector and a slope calculator used to determine glucose trend information and hypoglycemic and/or hyperglycemic events that are implemented by a processor configured to execute computer-readable instructions in accordance with embodiments described herein.

FIG. 6A illustrates a block diagram of a CGM system including a wearable device and an external device, wherein an event detector is implemented in the external device in accordance with embodiments described herein.

FIG. 6B illustrates a block diagram of a CGM system including a wearable device and an external device, wherein an event detector is implemented in the wearable device in accordance with embodiments described herein.

FIG. 7 illustrates graphs showing different embodiments of slope calculations and a corresponding graph of glucose concentrations in accordance with embodiments described herein.

FIG. 8A illustrates a graph showing projected glucose concentrations implemented as a cone of confidence in accordance with embodiments described herein.

FIG. 8B illustrates another graph showing projected glucose concentrations implemented as a cone of confidence in accordance with embodiments described herein.

FIG. 9 illustrates a flowchart showing a method of predicting an analyte concentration trend in accordance with embodiments described herein.

FIG. 10 illustrates a flowchart showing a method of predicting a glucose concentration trend in accordance with embodiments described herein.

### DETAILED DESCRIPTION

The apparatus, systems, and methods disclosed herein describe continuous analyte monitoring (CAM) systems and methods implemented as continuous glucose monitoring (CGM) systems, CGM methods, CGM displays, CGM display methods, and the like. The apparatus, systems, and methods disclosed herein may also be implemented to monitor and display other analytes (e.g., analyte concentrations), such as cholesterol, lactate, uric acid, alcohol, and other analytes.

In order to more closely monitor an individual's glucose (or other analyte) concentrations and detect shifts in glucose concentrations, apparatus, systems, and methods of continuous glucose monitoring (CGM) have been developed. The apparatus, systems, and methods described herein predict analyte (e.g., glucose) concentration trends and/or events (hypo or hyper). Some CGM systems may include a sensor portion (e.g., a biosensor) that is inserted under the skin of a user, and a non-implanted processing portion that is adhered to the outer surface of the skin, for example, the abdomen or the back of the upper arm. Some of the CGM systems described herein measure glucose concentrations in interstitial fluid or in samples of non-direct capillary blood. A processor executing computer-readable instructions calculates the glucose concentrations in the blood based on the measured glucose concentrations in the interstitial fluid. Other CGM systems may use optical and/or other sensors to generate data that is used to calculate glucose concentrations.

Some CGM systems predict hypoglycemic and hyperglycemic events, wherein hypoglycemic events may occur when glucose concentrations are less than a predetermined glucose concentration, and hyperglycemic events may occur when glucose concentrations are greater than a predetermined glucose concentration. In the examples described herein, hypoglycemic events occur when glucose concentrations are less than 70 mg/dl, and hyperglycemic events may occur when glucose concentrations are greater than 180 mg/dl. If a user is given 15-30 minutes advanced warning, for example, of a hypoglycemic or hyperglycemic event, the user may respond (e.g., with therapeutic measures) to rectify the glucose concentration issue and avoid hypoglycemic and/or hyperglycemic events altogether.

Some known CGM systems may predict hypoglycemic and hyperglycemic events based on several variables, such as exercise and dietary intake. These CGM systems require users to input exercise preformed and foods consumed, which may include portion sizes and calories consumed, for example, to predict hypoglycemic and hyperglycemic events. Because these known CGM systems require user input, they may not accurately predict glucose concentrations 15-35 minutes in the future so that users may avoid hypoglycemic and hyperglycemic events. For example, a user may not accurately enter exercises performed or foods consumed, or may not want to be bothered entering this information at all. In other situations, users' bodies react differently in response to certain exercises and foods, which may not be taken into consideration by these CGM systems. In addition, there may be other factors that affect glucose concentrations, but are not considered by these CGM systems.

The apparatus, systems, and methods described herein provide accurate glucose (and other analyte) concentration trend or behavior predictions using unique artificial intelligence models and inputs. For example, the artificial intelligence models may be trained using data from a plurality of individuals other than a present user. In some embodiments, the individuals undergo different activities during training that may affect glucose concentrations, such as consuming different foods and/or performing different activities. The artificial intelligence models (e.g., machine learning models) may identify trends in glucose concentrations and based on the trends predict, e.g., whether future glucose concentrations cross a hypo or hyper glycemic threshold. The CGM systems described herein may monitor previously calculated glucose concentrations of a user and input these previously calculated glucose concentrations into an artificial intelligence algorithm or model, which may then predict future glucose concentration trends or behaviors of the user. The user does not need to input foods consumed or exercise performed for the artificial intelligence model to predict such future trends or behaviors.

In accordance with some embodiments of the artificial intelligence models described herein, each calculated glucose concentration is related to its adjacent calculated glucose concentrations in short term and/or long-term relationships. Because of the continuous nature of CGM, prior calculated glucose concentrations may contain information relevant to predicting future glucose concentrations. That is, each calculated glucose concentration may be related to its adjacent (e.g., previous) calculated glucose concentrations, or even glucose concentrations calculated much earlier in time. For example, certain past glucose trends may be indicative of future glucose concentrations. The relationships of a present calculated glucose concentration to many previously-calculated glucose concentrations in a continuum have been found to be useful in predicting future glucose concentrations.

The glucose concentrations may also be used to determine the “slope” of present glucose concentrations (plotted in a graph). The slope of such glucose concentrations may inform a user of present and predicted directions (“trend information”) of glucose concentrations, which may be presented to the user via a display, e.g., of an external device in communication with a wearable CGM or CAM device. In some embodiments, the slope directions may be, for example, “rising,” “steady,” and “falling.” In other embodiments, the slope directions may be, e.g., “up fast,” “up slow,” “steady,” “down slow,” and “down fast.” Some glucose concentrations are calculated and/or displayed as a continuous glucose signal, which may be noisy. The methods and apparatus described herein may smooth the glucose signals during slope calculations, which provide more accurate slope calculations.

Methods and apparatus disclosed herein use artificial intelligence, such as machine learning models, to calculate slope of glucose and/or other analytes in a user. The methods and apparatus may use similar or identical data to calculate slope of the glucose signal. Therefore, slope calculations are more accurate than slope calculations of conventional CGM systems, which are prone to error due to noise sources.

The glucose trend or behavior predictions may include a certainty (e.g., a probability) that an event, such as a hypoglycemic event or a hyperglycemic event, will occur. The certainties may, in some embodiments, be functions of time. For example, the glucose trend or behavior predictions may be very certain in the short term, but may be less certain as a function of time. Some embodiments of the CAM systems and CGM systems disclosed herein display the analyte and/or glucose concentration trend or behavior predictions with confidence indications that indicate probabilities that hypoglycemic and/or hyperglycemic events will occur.

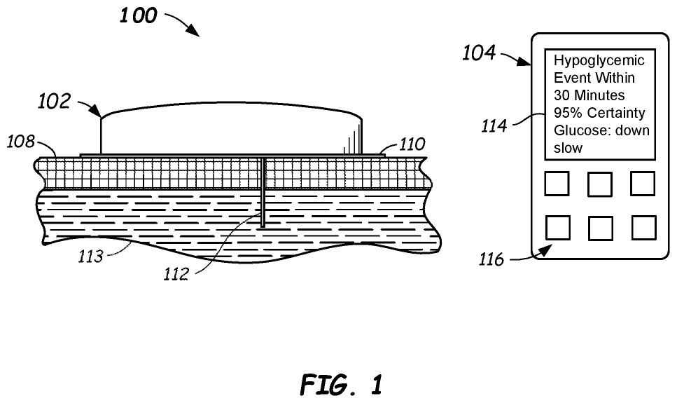

These and other methods, systems, and apparatus for predicting and displaying trends or behaviors of analyte (e.g., glucose) concentrations are described herein with reference to FIGS. 1-10.

FIG. 1 illustrates a block diagram of an embodiment of a continuous analyte monitoring system (CAM system) configured as a continuous glucose monitoring system (CGM system) 100. The CGM system 100 includes a wearable device 102 and an external device 104. Other types of CAM systems may be used with aspects of the following disclosure. The wearable device 102 may measure glucose concentrations in interstitial fluid, and the external device 104 may display the glucose concentrations, predicted glucose concentrations, trends in glucose concentrations, glucose concentration slopes, and/or other information. The wearable device 102 may be attached (e.g., adhered) to skin 108 of a user, such as by an adhesive 110.

The wearable device 102 may include a biosensor 112 that may be located subcutaneously in interstitial fluid 113 of a user and may directly or indirectly measure glucose concentrations in the interstitial fluid 113. The wearable device 102 may transmit the glucose concentrations to the external device 104, where the glucose concentrations, predicted glucose concentrations, and/or other information may be displayed on a display 114. The display 114 may be any suitable type of human-perceivable display, such as but not limited to, a liquid crystal display (LCD), a light-emitting diode (LED) display, or an organic light emitting diode (OLED) display.

The display 114 may display different formats of predicted glucose concentrations, such as individual numbers, graphs, and/or tables as described below. The display 114 may also display other information, such as trends in glucose concentrations. In the example embodiment of FIG. 1, the display 114 is shown displaying information related to a predicted hypoglycemic event and the present trend (“down slow”) of the user's glucose concentration. The display 114 may display different or additional data in other formats. In some embodiments, the external device 104 may include a plurality of buttons 116 or other input devices that enable users to select data and/or data formats displayed on the display 114. In some embodiments, the external device 104 may be a cellular telephone (e.g., a smart phone).

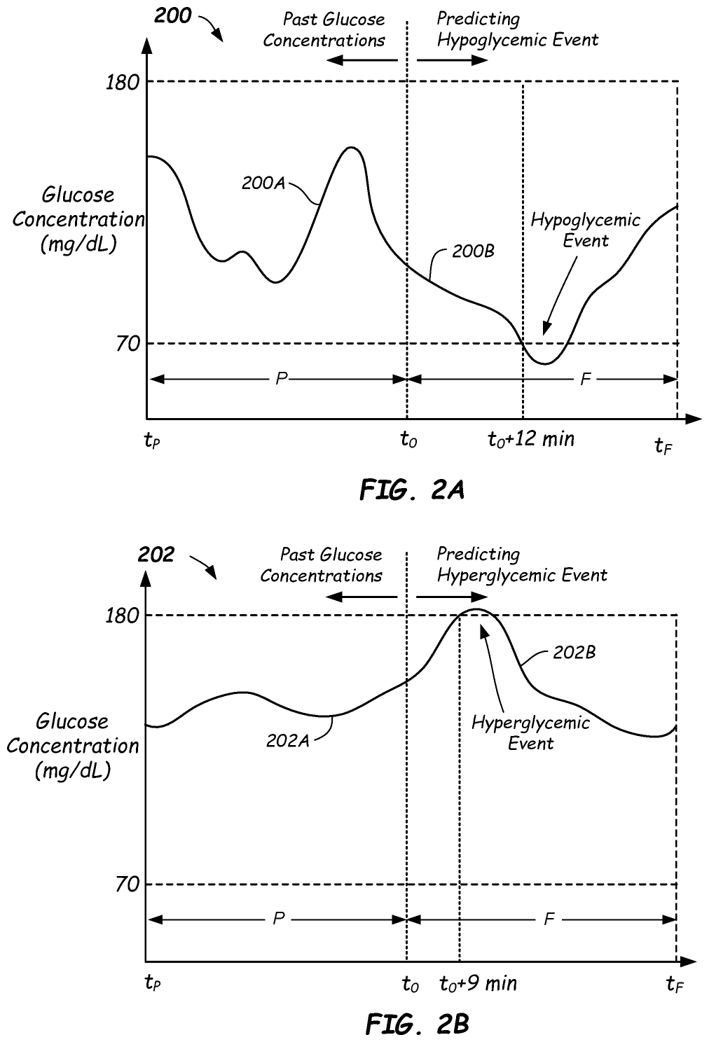

FIG. 2A illustrates a graph 200 showing an example of measured glucose concentrations including a hypoglycemic event of a user, and FIG. 2B illustrates a graph 202 showing an example of measured glucose concentrations including a hyperglycemic event of a user, each in accordance with embodiments described herein. A hypoglycemic event, as used herein, occurs when a user's glucose concentration falls below 70 mg/dl and a hyperglycemic event, as used herein, occurs when a user's glucose concentration rises above 180 mg/dl. Other predetermined analyte concentrations (e.g., predetermined glucose concentrations) may be indicative of hypoglycemic and hyperglycemic events. The glucose concentrations shown in FIGS. 2A and 2B are shown to describe processing and may or may not be displayed on display 114 (FIG. 1).

The graph 200 includes two parts, past glucose concentrations 200A of a user determined before a time t₀ and glucose concentrations 200B of the user determined after the time t₀. Glucose concentrations 200A and 200B may be calculated by a CGM system or a processor external to the CGM system, for example. CGM systems include systems that measure and/or calculate glucose concentrations in interstitial fluid via a probe located in the interstitial fluid, such as the CGM system 100. The CGM systems may include optical systems that optically measure and/or calculate users' glucose concentrations. The glucose concentrations may be obtained from other systems.

A time t₀ shown on the graph 200 represents a present time at which a present or most recent glucose concentration (or other analyte concentration) was processed (e.g., measured and/or calculated). For example, the CGM system 100 or an external processor may generate and/or receive data indicative of analyte concentration measurements, such as glucose concentration measurements and may calculate the present glucose concentration at the time t₀. The past glucose concentrations 200A are located to the left of the time t₀. As described herein, at least some of the past glucose concentrations 200A are processed by the machine learning model (or other artificial intelligence) to predict at time t₀ a future trend of glucose concentrations up to a future time t_{F} (e.g., to predict the trend of glucose concentrations 200B, which are shown to the right of time t₀) and, more particularly, to predict at time t₀ whether a hypoglycemic event will occur within the time period F, such as, e.g., the actual hypoglycemic event that occurs at a time t₀+12 minutes, as shown in FIG. 2A.

In some embodiments, the machine learning model may use a feedforward neural network with sixteen inputs, three hidden layers of twenty-four, ten, and five neurons each, and one output layer having a single output neuron. The single output neuron may be the certainty of an event at a given time. Other neural network architectures may be used such as neural networks having different numbers of hidden layers, different numbers of neurons per hidden layer, etc. Other artificial intelligence, trained models, and machine learning models may be used, such as gradient boosted regression trees (GBRT), linear regression, and random forests. Thus, training a model may include training a machine learning model and/or one of the above-listed models.

The graph 200 shows the past glucose concentrations 200A extending back to a time t_{P}, which may be P minutes less than t₀. In some embodiments, the period P may be about thirty minutes. However, the machine learning model may analyze past glucose concentrations from longer or shorter periods than thirty minutes. For example, the machine learning model may analyze the past glucose concentrations 200A back forty-five minutes from the time t₀, which may require substantial processing, but may provide accurate predicted glucose concentration trends. In some embodiments, the machine learning model may analyze the past glucose concentrations 200A back fifteen minutes from the time t₀, which may not provide as accurate predicted glucose concentration trends, but may require less processing.

The predicted glucose concentration trend may be based on or include a predicted strength or certainty (e.g., probability) that the predicted glucose concentration trend will actually occur as glucose concentrations 200B. Accordingly, predicted hypoglycemic events and hyperglycemic events may be based on the predicted strength or certainty that the predicted glucose concentration trend will occur as, e.g., glucose concentrations 200B. For example, the prediction of the hypoglycemic event may be based on at least a 95% certainty that the hypoglycemic event will occur within the time period F (which actually does occur at a time t₀+12 minutes, as shown in FIG. 2A).

FIG. 2B illustrates a graph 202 showing an example of past glucose concentrations 202A of a user determined before a time t₀ and glucose concentrations 202B of the user determined after the time t₀, which includes a hyperglycemic event, in accordance with embodiments described herein. The prediction of the hyperglycemic event may be calculated based on the same certainty that a predicted glucose concentration trend will occur as glucose concentrations 202B. The machine learning model analyzes the past glucose concentrations 202A (from the time t₀ back to the time t_{P}) to calculate the predicted glucose concentration trend to the time t_{F} and, more particularly, to predict at time t₀ whether a hyperglycemic event will occur within the time period F, such as, e.g., the actual hyperglycemic event that occurs at a time t₀+9 minutes, as shown in FIG. 2B. Based on the above example, the machine learning model may predict the hyperglycemic event occurring within the time period F with a 95% certainty.

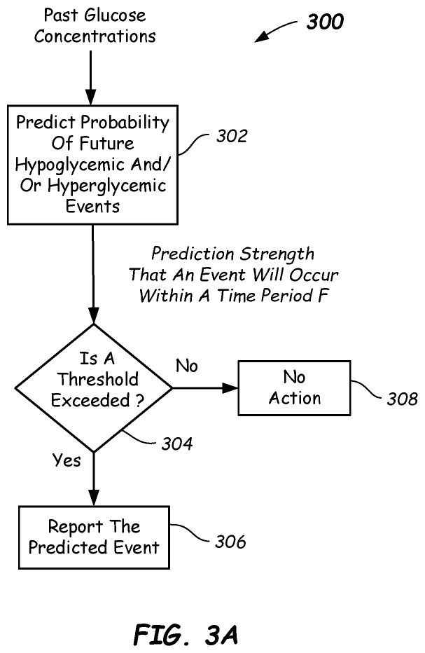

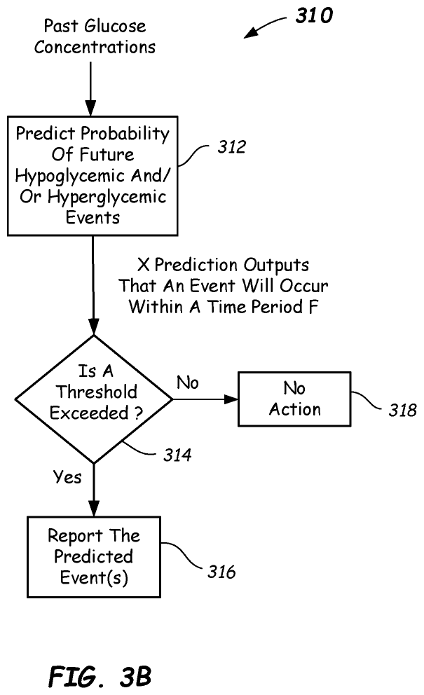

FIG. 3A illustrates an embodiment of a method 300 of predicting hypoglycemic events and/or hyperglycemic events. In block 302, past glucose concentrations are received and future hypoglycemic and/or hyperglycemic events are predicted based on the past glucose concentrations. The operations performed in block 302 may be performed using a machine learning model or other artificial intelligence algorithm as described herein. In some embodiments, the past glucose concentrations may be analyzed back to the time t_{P} (FIGS. 2A-2B), wherein the time t_{P} is the period P minutes prior to the time t₀. Higher values of the period P may use more processing time and/or resources than lower values of the period P to generate the output of block 302. However, analyzing more past glucose concentrations using a higher value of the period P may provide more accurate outputs from the block 302. Lower values of the period P may result in less accurate outputs of the block 302 compared to use of higher values of the period P, but using the lower values of the period P may use less processing time and/or resources than the higher values of the period P.

The output of block 302 includes a prediction strength (e.g., probability) that one or more hyperglycemic events and/or one or more hyperglycemic events will occur within a predetermined time period, such as within the time period F (FIGS. 2A-2B). In some embodiments, the time period F may be set by a user or another entity. In some embodiments, the prediction strength is a probability that a hypoglycemic event and/or a hyperglycemic event will occur within the time period F. In decision block 304 a determination is made as to whether the prediction strength exceeds a predetermined threshold. If the determination made in decision block 304 is positive, a report of the predicted event may be sent to a user per block 306. If the determination in decision block 304 is negative, no action may be taken per block 308.

In the following example, the past glucose concentrations 200A of the graph 200 of FIG. 2A are input to block 302. An event predictor (described below) related to block 302 determines that there is a certainty, such as a probability of 95%, that a hypoglycemic event will occur within the time period F. When the data generated in block 302 is input to the decision block 304 and the threshold certainty is set to 95%, the determination from decision block 304 is positive that a hypoglycemic event will occur within the time period F. This information of the predicted future hypoglycemic event may be reported to the user per block 306. For example, the display 114 (FIG. 1) or another reporting device may provide information of the future hypoglycemic event to the user or another person, such as a medical provider.

In some embodiments, the information may include the certainty (e.g., probability) of the hypoglycemic event and that the hypoglycemic event will occur within the time period F (e.g., within 30 minutes). In some embodiments, the information may include the expected time of the hypoglycemic event, which in the graph 200 of FIG. 2A is in twelve minutes. Similar information may be generated for the hyperglycemic event based on the past glucose concentrations of the graph 202 of FIG. 2B.

In embodiments where the threshold within the decision block 304 is set to greater than 95%, the probability of an event calculated in block 302 described above will not exceed the threshold. Accordingly, no action will be taken per block 308.

FIG. 3B illustrates a method 310 of predicting future hypoglycemic and/or hyperglycemic events. The processing of the method 310 may be more dynamic and may provide more information to the user than the method 300 of FIG. 3A. For example, the method 310 may be similar to the method 300, except the method 310 may generate a plurality (X) of outputs. Each of the X outputs may provide a certainty or probability that an event will occur for each of a plurality X of time increments in the future. The plurality of time increments or samples may be between the time t₀ and the time t_{F}, wherein each sample is N (e.g., N minutes) from a previous sample. Thus, a first output X1 may provide a probability, P1(t₀+N, t₀), that a hypoglycemic or a hyperglycemic event will occur at a first time t₀+1N relative to the time t₀. A second output X2 may provide a probability, P2(t₀+2N, t₀), that a hypoglycemic or a hyperglycemic event will occur at a second time t₀+2N relative to the time t₀. The number of outputs X may be equal to the time period F divided by the time between the samples N. In some embodiments, N is equal to three minutes. In other embodiments, N may be equal to increments between one minute and five minutes. In other embodiments, N may be equal to increments between two minutes and four minutes. In some embodiments, the time period F may be equal to thirty minutes and in other embodiments, the time period F may be equal to forty-five minutes. In yet other embodiments, the time period F may be equal to ten minutes or fifteen minutes. Other values of F, X, and N may be used. In some embodiments, the periods between the samples N may not be uniform.

The operations performed in block 312 may be performed using a machine learning model or other artificial intelligence algorithm as described herein. In some embodiments, the past glucose concentrations may be analyzed back the period P (minutes) from the time t_{P}. As described above, higher values of the period P may use more processing time than lower values of the period P to generate the outputs of block 312, but the outputs of block 312 may be more accurate. Lower values of the period P may result in less accurate outputs of the block 302 compared to use of higher values of the period P, but using the lower values of the period P may take less processing than the higher values of the period P.

As described above, the outputs of block 312 may include prediction certainties (e.g., probabilities) that a hypoglycemic event and/or a hyperglycemic event will occur at various times within the time period F. The time period F may be set by a user or another entity. In some embodiments, the time period F is 15 minutes, which may provide accurate results. In other embodiments, the time period F is 30 minutes, which may provide less accurate results, but provides the user with a longer time frame within which to take any necessary action. The time period F may be of other durations, such as, e.g., forty-five minutes.

In decision block 314 determinations are made as to whether one or more of the probabilities exceeds a threshold. If the determination made in decision block 314 is positive, one or more reports (e.g., alerts) may be sent or reported to a user per block 316. The one or more reports may include information as to when the events are expected to occur and the certainties (e.g., probabilities) that the events will occur. For example, if the threshold is set at a 95% certainty, the user may be notified if one of the outputs predicts that a hypoglycemic or hyperglycemic event will occur with at least 95% certainty and when the event(s) will occur. If the determination in decision block 314 is negative, no hypoglycemic and/or hyperglycemic events have been predicted and no action may be taken per block 318.

When the past glucose concentrations 200A of the graph 200 of FIG. 2A are input to block 312, the event predictor related to block 312 determines certainties or probabilities that hypoglycemic events and/or hyperglycemic events will occur at different times between the time t₀ and the time t_{F}. For example, when N equals three minutes, block 312 outputs a probability of a hypoglycemic or hyperglycemic event for every three-minute interval between time t₀ and time t_{F}. In some embodiments, the interval times N may not be equal, so the outputs may occur at differing intervals.

When the data generated in block 312 is input to the decision block 314, the decision block 314 determines whether any of the probabilities exceed a predetermined threshold. If so, then processing proceeds to block 316 where the user is notified of the predicted event(s). The user may be notified of the time of the pending event(s) and, in some embodiments, the certainty that the event(s) will occur. For example, referring to the graph 200 of FIG. 2A, if the certainty threshold in decision block is 95%, the user may be informed that a hypoglycemic event is likely to occur in twelve minutes and there is a 95% certainty that the hypoglycemic event will occur. Decision block 314 may output a plurality of reports for times in which the hypoglycemic event is predicted to occur.

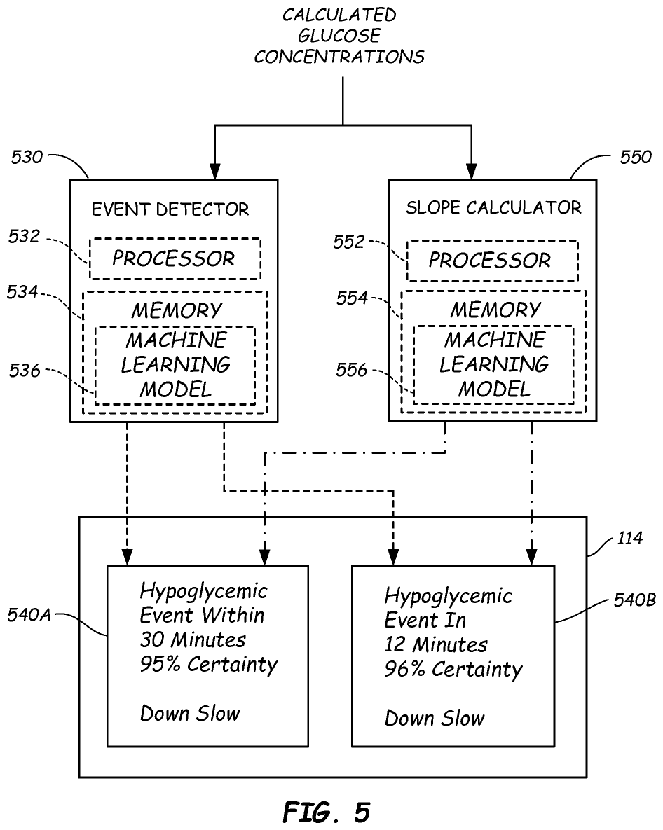

An event detector (e.g., event detector 530 of FIG. 5 described further below) operating in block 302 and block 312 may include artificial intelligence, such as a machine learning model, that may be trained by prior analysis of a plurality of individuals. For example, glucose concentrations of a plurality of individuals may be monitored and/or analyzed to associate past glucose concentration trends with future glucose concentration trends. Such training using the individuals' glucose concentration trends enables a prediction of a future hypo or hyper glycemic event without the user spending time, which can be excessive, training a unique machine learning model. In addition, by training the machine learning model using a plurality of individuals, the machine learning model may be trained based on a variety of different glucose concentration trends that the user could likely not provide. For example, the individuals may have undergone different exercises and had different dietary intakes than the user could undergo during a training period. Thus, the machine learning model trained by analyzing glucose concentrations of a plurality of individuals may be more accurate than a machine learning model trained solely based on a single user's glucose concentration history.

In some embodiments, the machine learning model is trained by receiving or analyzing past glucose concentrations of the individuals during various periods and correlating the past glucose concentrations with future glucose concentrations. In some embodiments, the past glucose concentrations may be received or analyzed at regular increments, such as every three minutes. Other increments, such as every two minutes or every four minutes may be used. The periods of time that the past glucose concentrations are calculated and/or measured may be long enough to develop trends to train the machine learning model. In some embodiments, the periods of time may be thirty minutes. In other embodiments, longer periods, such as forty-five or sixty minutes may be used to gather more information on glucose concentration trends.

Referring to FIG. 3A, the machine learning model may analyze past glucose concentrations 200A to learn how the past glucose concentrations affect future glucose concentrations. For example, certain waveforms in the past glucose concentrations 200A may cause the future glucose concentrations to be at certain levels at certain times. Based on this analysis, the machine learning model may predict the glucose concentrations of the user.

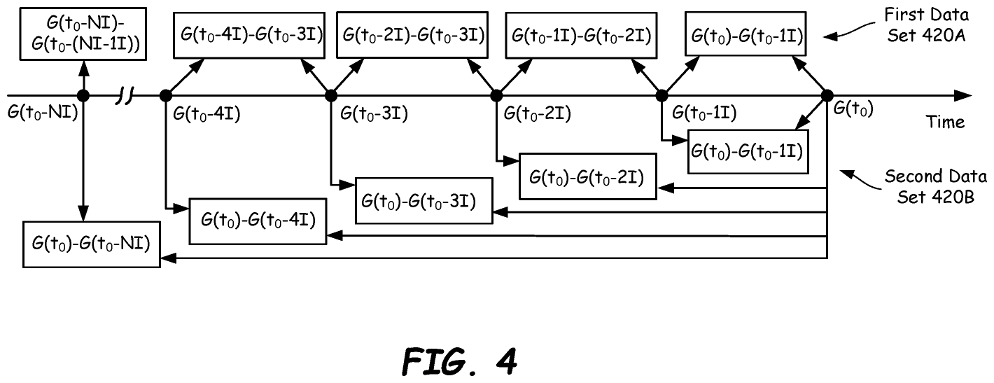

FIG. 4 is a block diagram showing glucose concentration calculations on a timeline that may be received or calculated by the event detector (e.g., event detector 530 of FIG. 5). These glucose calculations may be input to the machine learning model. The glucose concentrations on the timeline may be received from a CGM, such as the CGM system 100 (FIG. 1) attached to a user. The glucose concentrations from a present glucose concentration G(t₀) (e.g., a current analyte concentration G(t₀)) measured at time t₀ back in time t₀ a glucose concentration G(t₀-NI) are analyzed, wherein N is a sample number and I is a time period between samples. The glucose concentration G(t₀-NI) may occur at time t_{P}, such that the period P shown in graph 200 and graph 202 in FIGS. 2A and 2B, respectively, is equal to NI. Other values of the period P may be used. Thus, the event detector may receive a plurality of analyte (e.g., glucose) concentration measurements at measurement times between the time t₀ of a most recent analyte concentration measurement and the time t_{P}.

The differences in analyte concentrations (e.g., glucose concentrations) calculated by the event detector may be referred to as a first data set 420A and a second data set 420B. The first data set 420A includes a plurality of incremental differences in glucose concentrations. For example, the first data set 420A includes the glucose concentration differences: G(t₀-NI)−G(t₀-1I); G(t₀-1I)−G(t₀-2I); G(t₀-2I)−G(t₀-3I); G(t₀-3I)−G(t₀-4I) . . . to G(t₀-NI)−G(t₀-(NI-1I)). Thus, calculating the first data set 420A may include calculating differences in analyte (e.g., glucose) concentrations between consecutively measured analyte concentrations between the time t_{P} and the time t₀.

The second data set 420B may include differences in glucose concentrations all referenced from the most recently measured glucose concentration G(t₀). For example, the second data set 420B set includes the glucose concentration differences: G(t₀)−G(t₀-1I); G(t₀)−G(t₀-2I); G(t₀)−G(t₀-3I); G(t₀)−G(t₀-4I) . . . to G(t₀)−G(t₀-NI). Thus, calculating the second data set 420B may include calculating differences in analyte (e.g., glucose) concentrations between the analyte concentration G(t₀) at the time t₀ and analyte concentrations at measurement times before the time t₀. In some embodiments, the machine learning model of the event detector may be trained at least in part based on data of the first data set 420A and the second data set 420B from the individuals used to train the machine learning model. In some embodiments, the machine learning model may be trained by further analyzing glucose concentrations of the user.

FIG. 5 illustrates an embodiment of an event detector 530 implemented by components including a processor 532. The event detector 530 also includes memory 534 that may store the machine learning model 536 or other artificial intelligence that performs the functions described herein. The memory 534 and other memory within the CGM system 100 (FIG. 1) may be any suitable type of memory, such as, but not limited to, one or more of a volatile memory and/or a non-volatile memory capable of storing code of algorithms (e.g., machine learning models) described herein. The machine learning model 536 may be an algorithm comprising computer-readable instructions stored in the memory 534 that, when executed by the processor 532, cause the processor 532 to predict one or more glucose concentration trends based on previously calculated glucose concentrations as described herein.

When performing the method 300 of FIG. 3A and the method 310 of FIG. 3B, the event detector 530 may output the aforementioned probabilities related to predicting glucose concentrations. The event detector 530 and/or the processor 532 may be directly or indirectly coupled to one or more displays (e.g., display 114 of FIG. 1) that display predicted glucose concentrations for a user or other person or entity. The display 114 may also display other information as described herein. In the embodiment of FIG. 5, a first display embodiment 540A shows example information that may be displayed on the display 114 in response to processing the past glucose concentrations 200A (FIG. 2A) per the method 300 of FIG. 3A. For example, the first display embodiment 540A indicates that there is a likelihood that a hypoglycemic event will occur within the time period F, which in the example of FIG. 5 is 30 minutes. The first display embodiment 540A may also show the prediction certainty (e.g., probability) that the hypoglycemic event will occur, which in the example of FIG. 5 is 95%.

A second display embodiment 540B shows example information that may be displayed on the display 114 in response to processing the past glucose concentrations 200A (FIG. 2A) per the method 310 of FIG. 3B. For example, the second display embodiment 540B may indicate when the hypoglycemic event is predicted to occur and the prediction certainty (e.g., probability) used to make the determination. In the example of FIG. 5, there is a likelihood that a hypoglycemic event will occur in twelve minutes based on a 96% prediction certainty. The second display embodiment 540B may also display information related to other predicted glycemic events.

In addition to the foregoing display embodiments, the display 114 may also display portions of the graph 200 (FIG. 2A) and the graph 202 (FIG. 2B). In some embodiments, the display 114 may display at least a portion of the predicted glucose concentrations 200B and/or at least a portion of the past glucose concentrations 200A. In other embodiments, the display 114 may display at least a portion of the predicted glucose concentrations 202B and/or at least a portion of the past glucose concentrations 202A.

The first display embodiment 540A and/or the second display embodiment 540B may be displayed in any of a plurality of locations. In some embodiments, the first display embodiment 540A and/or the second display embodiment 540B may be displayed on the display 114 (FIG. 1) of the CGM system 100. In other embodiments, the first display embodiment 540A and/or the second display embodiment 540B may be displayed on a device external to a CGM system, such as display devices used by a medical provider or the like. For example, the first display embodiment 540A and/or the second display embodiment 540B may be displayed on diagnostic equipment, such as in a hospital or the like.

In some embodiments, the processor 532 may inform the user of a predicted hypoglycemic event and/or a hyperglycemic event via an audio signal and/or a tactile signal. The audio signal may be a voice informing the user of the information in the first display embodiment 540A and/or the second display embodiment 540B. Other audio signals, such as alarms may be used. The tactile signal may provide the information in Braille or other tactile formats, such as vibration of the external device 104.

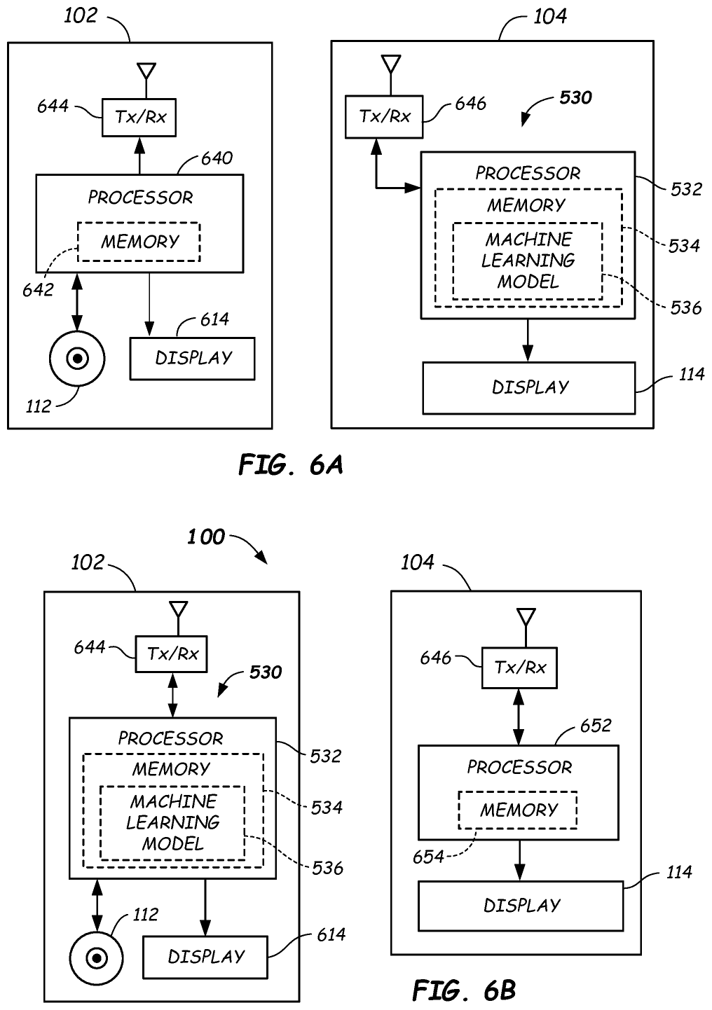

FIG. 6A illustrates a block diagram of an example of the CGM system 100 including the wearable device 102 and the external device 104, wherein the event detector 530 is implemented in the external device 104. In the embodiment of FIG. 6A, the wearable device 102 may include a processor 640 that may be electrically coupled to the biosensor 112. The processor 640 may transmit signals to and receive signals from the biosensor 112. At least one of the signals received from the biosensor 112 is indicative of a glucose concentration in interstitial fluid of a user. The processor 640 and/or memory 642 located in the wearable device 102 may include instructions that, when executed by the processor 640, cause the processor 640 to process data received from the biosensor 112. In some embodiments, the instructions may cause the processor 640 to calculate glucose concentrations of the user based at least in part on the signal received from the biosensor 112. In other embodiments, the instructions may cause the processor 640 to convert the data received from the biosensor 112 and/or the calculated glucose concentrations into a format for transmission from the wearable device 102 by way of a transceiver 644.

In the embodiment of FIG. 6A, the external device 104 may include a transceiver 646 that may receive the data transmitted from the wearable device 102. Accordingly, the wearable device 102 and the external device 104 may be communicatively coupled. In some embodiments the communicative coupling of the wearable device 102 and the external device 104 may be by way of wireless communication via the transceiver 644 and the transceiver 646. Such wireless communication may be by any suitable means including but not limited to standards-based communications protocols such as the Bluetooth® communications protocol. In various embodiments, wireless communication between the wearable device 102 and the external device 104 may alternatively be by way of near-field communication (NFC), radio frequency (RF) communication, infra-red (IR) communication, or optical communication. In some embodiments the wearable device 102 and the external device 104 may be communicatively coupled by one or more wires. In some embodiments, the external device 104 may be a server or the like and the communication between the wearable device 102 and the external device 104 may be via the Internet.

The transceiver 646 may be electrically coupled to the event detector 530. In some embodiments where the glucose concentrations are calculated by the processor 640 in the wearable device 102, the event detector 530 may function in a similar manner as described in FIG. 5. In embodiments where the glucose concentrations are not calculated in the wearable device 102, the memory 534 may store instructions that, when executed by the processor 532, cause the processor 532 to calculate the glucose concentrations. These calculated glucose concentrations are then processed by the event detector 530 as described herein to predict hypoglycemic events and/or hyperglycemic events as described herein.

In the embodiment of FIG. 6A, the event detector 530 may predict hypoglycemic and/or hyperglycemic events as described herein. In some embodiments, the event detector 530 may also calculate slopes as well as trends of glucose concentrations of the user as described herein. In some embodiments, the event detector 530 does not use slope information to make its predictions, while in other embodiments, the event detector 530 may receive slope information from, e.g., a separate software module (e.g., a slope calculator) and may use that slope information to make its predictions. The event detector 530 may output the predictions to the display 114. In some embodiments, the event detector 530 may output at least the first display embodiment 540A and/or the second display embodiment 540B of FIG. 5 to the display 114. In some embodiments, the transceiver 646 may output the predicted hypoglycemic events and/or hyperglycemic events to other devices, such as other externals devices (not shown) or a server (not shown), such as a server coupled to a computer of a medical provider.

FIG. 6B illustrates a block diagram of the CGM system 100 including the wearable device 102 and the external device 104, wherein the event detector 530 is implemented in the wearable device 102. In the embodiment of FIG. 6B, the memory 534 may include instructions that, when executed by the processor 532, cause the processor 532 to calculate glucose concentrations in response to signals received from the biosensor 112. The calculations of glucose concentrations may be performed on other processors.

The event detector 530 may receive the glucose concentrations and predict hypoglycemic and/or hyperglycemic events as described above. The predictions may be transmitted to the external device 104 by way of the transceiver 644. The external device 104 may receive the predictions by way of the transceiver 646 and may display the predictions on the display 114 as described herein. In the embodiment of FIG. 6B, the external device 104 may include a processor 652 and memory 654 wherein the memory 654 may store instructions that, when executed by the processor 652, cause the information received from the event detector 530 to be displayed on the display 114. In the embodiment of FIG. 6B, the wearable device 102 and/or the external device 104 may output the predictions to other devices, such as other externals devices or a server (none shown).

In the embodiments of FIGS. 6A and 6B, the wearable device 102 may include a display 614 that may display the data and information described with regard to the display 114. The display 614 may be any suitable type of human-perceivable display, such as, but not limited to, a liquid crystal display (LCD), a light-emitting diode (LED) display, or an organic light emitting diode (OLED) display.

In some embodiments, the user or another entity may select the threshold for the certainty or probability that hypoglycemic events and/or hyperglycemic events are detected (e.g., detection rates). Higher thresholds may yield lower detection rates and lower false alarm rates than with lower thresholds. Lower thresholds, however, may provide a higher number of earlier warnings of hypoglycemic events and hyperglycemic events, but may have higher false alarm rates. Thus, there is a tradeoff between earlier warnings and receiving more false alarms. In some embodiments, the user may be given options to choose a threshold that the user is comfortable with while still presenting information about the probability of events occurring in the future.

Tables 1 and 2 below each show example performances of outputs of a trained model, such as a trained machine learning model, having different thresholds for detecting hypoglycemic and/or hyperglycemic events. Table 1 shows an example analysis using a high threshold (e.g., 95%), and Table 2 shows the analysis with the same data, but using a lower threshold (e.g., 90%).

TABLE 1
Trained model results using high threshold

| | Output Number (X) | | | | | | | | | | |
|---|---|---|---|---|---|---|---|---|---|---|---|
| | 0 | 1 | 2 | 3 | 4 | 5 | 6 | 7 | 8 | 9 | 10 |
| Detect | 90.1 | 93.2 | 95.7 | 96.9 | 97.6 | 98.0 | 98.4 | 98.8 | 99.0 | 99.0 | 99.1 |
| Rate (%) | | | | | | | | | | | |
| False | 0.14 | 0.18 | 0.23 | 0.32 | 0.39 | 0.45 | 0.51 | 0.57 | 0.62 | 0.66 | 0.69 |
| Alarm | | | | | | | | | | | |
| Rate (%) | | | | | | | | | | | |
| Average | 17.4 | 18.4 | 20.4 | 22.3 | 24.1 | 25.8 | 27.0 | 28.0 | 28.9 | 29.5 | 29.8 |
| Advance | | | | | | | | | | | |
| Notice | | | | | | | | | | | |
| (minutes) | | | | | | | | | | | |

TABLE 2
Trained model results using lower threshold

| | Output Number (X) | | | | | | | | | | |
|---|---|---|---|---|---|---|---|---|---|---|---|
| | 0 | 1 | 2 | 3 | 4 | 5 | 6 | 7 | 8 | 9 | 10 |
| Detect | 95.3 | 96.5 | 97.5 | 98.5 | 99.2 | 99.5 | 99.6 | 99.7 | 99.8 | 99.9 | 100 |
| Rate (%) | | | | | | | | | | | |
| False | 0.24 | 0.32 | 0.43 | 0.58 | 0.72 | 0.86 | 1.00 | 1.13 | 1.23 | 1.26 | 1.26 |
| Alarm | | | | | | | | | | | |
| Rate (%) | | | | | | | | | | | |
| Average | 20.6 | 22.5 | 25.5 | 28.3 | 30.5 | 32.7 | 34.5 | 36.0 | 37.2 | 38.2 | 39.0 |
| Advance | | | | | | | | | | | |
| Notice | | | | | | | | | | | |
| (minutes) | | | | | | | | | | | |

As described herein, the CGM system 100 (FIG. 1) may provide indications of analyte concentration (e.g., glucose concentration) trends to the user. In some embodiments, the indications may include “up fast,” “up slow,” “stable,” “down slow,” and “down fast.” The CGM system 100 may use other glucose concentration trend indications. Embodiments described herein use artificial intelligence, such as machine learning models, to estimate or calculate future slope S(t) and/or the present slope S(t₀). The future slope S(t) may be calculated to the time t_{F} (FIGS. 2A-2B), which may be F minutes into the future from time t₀. The time t_{F} may, in some examples, be fifteen minutes from the time t₀. In other embodiments, the time t_{F} may be other times in the future, such as ten minutes or forty-five minutes. Based on the slope S(t), a processor or the like may cause the display 114 (FIG. 1) to display glucose concentration trend indications to the user.

In some embodiments, the machine learning model used to calculate slope may use a feedforward neural network with sixteen inputs, three hidden layers of twenty-four, ten, and five neurons each, and one output layer having a single output neuron. The single output neuron may be the slope S(t) at a given time. Other neural network architectures may be used such as neural networks having different numbers of hidden layers, different numbers of neurons per hidden layer, etc. Other artificial intelligence, trained models, and machine learning models may be used, such as gradient boosted regression trees (GBRT), linear regression, and random forests.

Returning to FIG. 5, a slope calculator 550 is illustrated that may be implemented within or used by the CGM system 100 (FIG. 1). In some embodiments, e.g., the slope calculator 550 may be external to the CGM system 100. The slope calculator 550 may estimate or calculate present slope S(t₀) and/or future slope S(t). In some embodiments, the slope calculator 550 may include a processor 552 that executes instructions stored in a memory 554.

The memory 554 may store the machine learning model 556 or other artificial intelligence as described above that calculates the slope S(t) based at least in part on past glucose concentrations. The slope calculations may also predict slopes of the predicted glucose concentrations. Accordingly, the slope calculations may be based on past glucose concentrations to project into the future. The slope calculator 550 may output the slope S(t) or an indication of the slope S(t), such as to the display 114. In the embodiment of FIG. 5, the slope calculator 550 has output an indication that the slope is trending slowly down.

The input to the machine learning model 556 shown in FIG. 5 may be solely past glucose concentrations that may be measured or calculated. In some embodiments, the first data set 420A and/or the second data set 420B of FIG. 4 may be the only inputs to the machine learning model 556 used to calculate the slope S(t). In some embodiments, the machine learning model 556 uses only the first data set 420A or the second data set 420B as inputs to calculate the slope S(t). Using the first data set 420A or the second data set 420B may provide faster slope calculations and may require less processing. In other embodiments, the machine learning model 556 uses both the first data set 420A and the second data set 420B to calculate the slope S(t), which may provide a more accurate slope calculation, but may require more processing. In other embodiments, the machine learning model 556 may use the first data set 420A and/or the second data set 420B and other inputs to calculate the slope S(t).

The first data set 420A and the second data set 420B may be based on glucose concentrations that go back a period P to the time t_{P}, which may be, e.g., twenty-four minutes from the present time t₀. Using the period P of twenty-four minutes may enable accurate slope calculations without overloading processors that execute the machine learning model 556 or other artificial intelligence. Other time periods for the period P may be used, such as fifteen minutes, thirty minutes, or forty-five minutes.

In some embodiments, the machine learning model 556 may be stored in the memory used to store other programs and may be executed on another processor. For example, the machine learning model 556 may be stored in the memory 534 and executed on the processor 532. In other embodiments, the machine learning model may be stored and executed on a computer or the like that is external to the CGM system 100 (FIG. 1). In some embodiments, the slope calculator 550 may be implemented in either or both the wearable device 102 or the external device 104 (FIGS. 6A-6B).

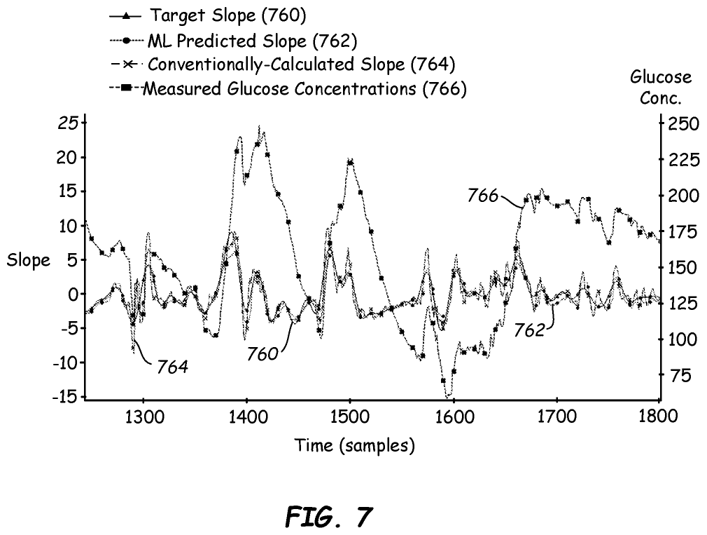

FIG. 7 illustrates graphs showing different embodiments of slope calculations and a corresponding graph of a user's measured glucose concentrations 766. In some embodiments, the measured glucose concentrations 766 may be made and reported by the CGM system 100 (FIG. 1). The graph of measured glucose concentrations 766 is marked with squares. The graph of the target slope 760 is marked with triangles and is the actual slope of the measured glucose concentrations 766.

A conventional slope measuring system has generated the conventionally-calculated slope 764, which is marked with x's. As shown in FIG. 7, the conventionally-calculated slope 764 is noisy and jumps erratically. Glucose trends calculated based on the conventionally-calculated slope 764 indicate sharp increases and decreases in glucose concentrations, which are erroneous. Accordingly, the user may take unnecessary and/or erroneous mitigation efforts to avoid glycemic events when relying on the conventionally-calculated slope 764.

A graph of machine learning (ML) predicted slope 762 is marked with circles and is generated by the machine learning model 536 (FIGS. 6A-6B) described herein. As shown in FIG. 7, the ML predicted slope 762 is smoother than the conventionally-calculated slope 764. In addition, the ML predicted slope 762 more closely follows the target slope 760 than the conventionally-calculated slope 764. Accordingly, the ML predicted slope 762 provides more accurate glucose concentration trends to the user than the conventionally-calculated slope 764.

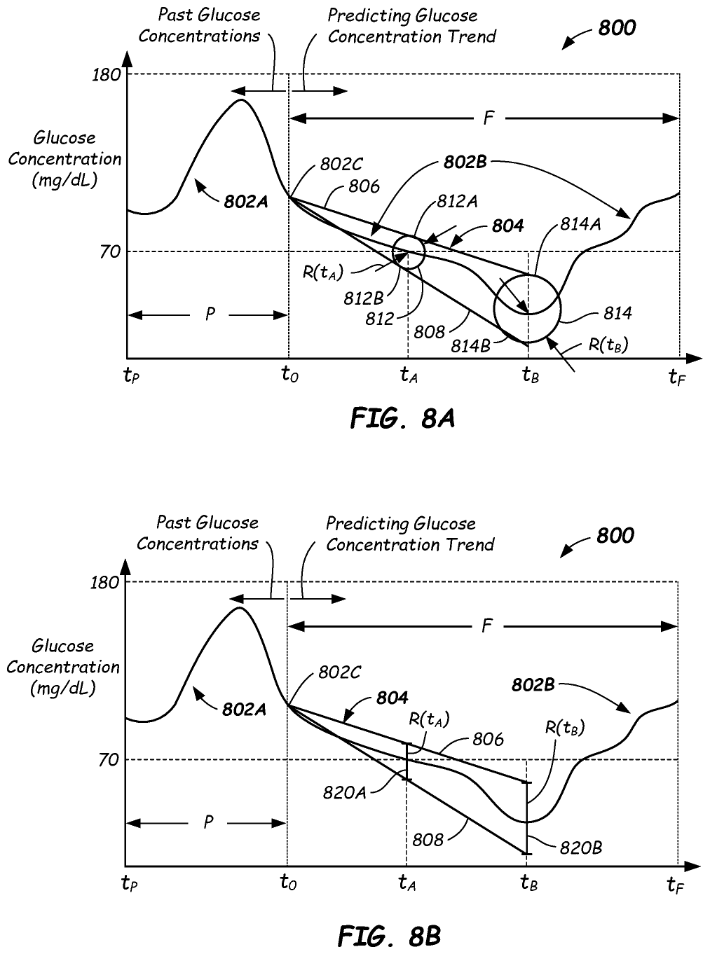

FIG. 8A illustrates a graph 800 showing glucose concentrations 802A and 802B and a cone of confidence 804. The cone of confidence 804 may be or include an indicium or graphic indicating a confidence or probability that a projected range of future glucose concentrations (e.g., projected range of analyte concentrations) will occur. The graph 800 includes past glucose concentrations 802A of a user determined by a CGM system before a time t₀ and glucose concentrations 802B of the user determined by the CGM system after the time t₀. In some embodiments, the glucose concentrations 802B may be displayed on the display 114 (FIG. 1), and in other embodiments, the past glucose concentrations 802A may also be displayed on the display 114. The time t₀ in the graph 800 of FIG. 8A is the time at which the cone of confidence 804 is determined. The cone of confidence 804 may show possible variations in the projected range of glucose concentrations occurring after the time t₀. The glucose concentrations 802B appearing within the cone of confidence 804 from the time t₀ to the time t_{B} indicates that the cone of confidence 804 had been accurately projected.

The cone of confidence 804 may include a first line 806 and a second line 808, which, in some embodiments, are boundaries of the cone of confidence 804. As described herein, the cone of confidence 804 may provide a user with a visual indication of the probabilities that projected glucose concentrations will occur as glucose concentrations 802B. As shown in FIG. 8A, the first line 806 and the second line 808 may converge at a convergence point 802C on the graph 800 at the time t₀. The first line 806 and the second line 808 may diverge from each other as a function of time. The vertical distance between the first line 806 and the second line 808 represents a degree of confidence in the projected glucose concentrations as a function of time. For example, the projected glucose concentrations may show the most likely future glucose concentration measurements. The area bounded by the first line 806 and the second line 808 include possible other future glucose concentration measurements.

The cone of confidence 804 enables users to quickly visualize confidence of the projected glucose concentrations. For example, the cone of confidence 804 enables users to visualize likelihoods of glycemic events in the future. In the embodiment described in FIG. 8A, the cone of confidence 804 extends to a time t_{B}. In other embodiments, the cone of confidence 804 may extend to the time t_{F} (i.e., time period F). In the embodiment of FIG. 8A, the cone of confidence 804 indicates that there is virtually no probability of a glycemic event in the very near future (e.g., before time t_{A}). The cone of confidence 804 indicates that there is about a 50% likelihood of a hypoglycemic event at time t_{A}, which may, as an example, be fifteen minutes into the future. In other embodiments, the time t_{A} may be between ten minutes and twenty minutes into the future. The cone of confidence 804 also indicates that there is a very high likelihood of a hypoglycemic event at time t_{B}, which may, as an example, be thirty minutes into the future.

In the embodiment of FIG. 8A, the divergence of the first line 806 and the second line 808 may be calculated based on radii of circles centered about points on the graph of the projected glucose concentrations. In the embodiment of FIG. 8A, two circles, a first circle 812 and a second circle 814 have been calculated. The first line 806 extends from the convergence point 802C to an upper tangent 812A of the first circle 812 and to an upper tangent 814A of the second circle 814. The second line 808 extends from the convergence point 802C to a lower tangent 812B of the first circle and to a lower tangent 814B of the second circle 814. In the embodiment of FIG. 8A, the first line 806 and the second line 808 converge at the convergence point 802C, so the projected glucose concentrations may be outside the cone of confidence 804 proximate the convergence point 802C. In some embodiments, the first line 806 and the second line 808 may not converge at the time t₀. Depending on the shape of the graph of the past glucose concentrations 802A, the first line 806 and/or the second line 808 may not be straight.

Different methods may be employed to calculate or generate the cone of confidence 804. In some embodiments, the cone of confidence 804 is calculated using probabilities of future hypo and/or hyper glycemic events, present slope, and current and projected glucose concentrations. The probabilities of future glycemic events may be received from the event detector 530 (FIG. 5), for example. The slope may be received from the slope calculator 550 (FIG. 5), for example. The current glucose concentration G(t₀) may be generated by calculation or measurements from the CGM system 100. Other methods of predicting future glycemic events and calculating slope may be used to generate the cone of confidence 804.

The following describes an embodiment of generating the cone of confidence 804. Other methods may be used to generate the cone of confidence 804. In embodiments where the event detector 530 (FIG. 5) predicts a single glycemic event, a probability P(t_{A}, t₀) is calculated as the probability that a glycemic event will occur within the time t₀ relative to t₀. Such data may be used to generate a cone of confidence having a single circle, indicator, or graphic.

In embodiments where the event detector 530 (FIG. 5) provides probabilities of glycemic events for a plurality of future times, probabilities for each of the plurality of future times may be used to generate the cone of confidence 804. In such embodiments, a cone of confidence, such as the cone of confidence 804 having a plurality of circles or other confidence indicators, indicia, or graphics may be generated. In the embodiment of FIG. 8A, probabilities P(t_{A}, t₀) and P(t_{B}, t₀) may be used to generate the cone of confidence 804. When probabilities are determined at a number N times from the time t₀, the probabilities may be referred to as P(t_{N}, t₀).

For each probability P_{N}(t_{N}, t₀), the value of t on the graph, which may be referred to as the value X (value on the x-axis of the graph 800), is equal to t₀+t_{N}. The glucose concentration, which may be referred to as Y, is equal to G(t₀)+slope(t₀)*(t_{N}/T_{I}), wherein T_{I} is the period between time intervals t_{N}. For example, in the embodiment of FIG. 8A, to may be 15 minutes from t₀, and t_{B} may be thirty minutes from t₀, so T_{I} is equal to 15 minutes. The radii, R(t_{A}) and R(t_{B}) of circles 812, 814 in the cone of confidence 804 are equal to ABS([Y−G_{EVENT}])*(1−P_{N})*F, wherein G_{EVENT} is the glucose concentration (on the Y axis) that triggers a glycemic event. The radii may also be referred to as the deviation R(t_{A}) and R(t_{B}), e.g., that provides indications of the confidence or probability that the projected analyte concentration or projected glucose concentration is within a range. For example, in the embodiments, described herein, G_{EVENT} equals 70 mg/dl for a hypoglycemic event and 180 mg/dl for a hyperglycemic event. F is a scale factor (e.g., 3) that determines a scale factor of the circles 812, 814 and ABS( ) is absolute value. In some embodiments, the radii are bounded for aesthetic purposes. For example, the radii may be bounded from five to seventy-five, for example. Other formulas may be used to calculate circles or other graphics and indicia on the graph 800.

The following provides an example of generating a cone of confidence, such as the cone of confidence 804. In the following example, the present glucose concentration G(t₀) has been measured or calculated to be 100 mg/dl. The glucose concentration for a hypoglycemic event is 70 mg/dl, so G_{EVENT} is equal to 70. The event detector 530 and/or methods thereof have predicted a 70% chance of a hypoglycemic event occurring in fifteen minutes and a 90% chance of a hypoglycemic event occurring in thirty minutes. Thus, P_{A}(15, t₀) is equal to 0.7, P_{B}(30, t₀) is equal to 0.9, and the interval I is equal to 15 minutes. Based on the foregoing, t_{A} is equal to 15 and t_{B} is equal to 30. G(t_{A}), which corresponds to the projected glucose concentration at time t_{A}, is equal to 100-25(15/15), which equals 75. G(t_{B}), which corresponds to the projected glucose concentration at time t_{B}, is equal to 100-25(30/15), which equals 50. Based on the foregoing, the center of the first circle 812 or other indicator or graphic is at time (x-axis) of 15 minutes and glucose concentration (y-axis) of 75. The center of the second circle 814 or other indicator is at time (x-axis) of 30 minutes and glucose concentration (y-axis) of 50. The radius R(t_{A}) of the first circle or other indicator, which may be referred to as the distance R(t_{A}), is equal to (75−70)*(1−0.7)*5, wherein F is equal to 5, so the radius R(t_{A}) is equal to 7.5. The radius R(t_{B}) of the second circle 814 or other indicator, which may be referred to as the distance R(t_{B}), is equal to |(50−70)|*(1−0.9)*5, wherein F is equal to 5, so the radius R(t_{A}) is equal to 12.5.

The cone of confidence 804 may use indicators other than circles, such as ellipses or vertical lines. An embodiment of an ellipse may have a vertically-extending major axis twice the distance R(t_{A}) and centered at a point indicative of G(t_{A}). Referring now to FIG. 8B, the cone of confidence 804 includes a first vertical line 820A at the time t_{A} and a second vertical line 820B at the time t_{B}. The first vertical line 820A and the second vertical line 820B may have lengths that are calculated in the same or similar manner as the radii R(t_{A}), R(t_{B}) of the first circle 812 and the second circle 814, respectively. For example, the first vertical line 820A and the second vertical line 820B may have lengths that are twice the radii calculated for the first circle 812 and the second circle 814, respectively.

As described above, the graphics and indicium in the cone of confidence may have many forms. In some embodiments, R(t_{A}) is represented by a distance and at least one indicium is displayed that includes at least one graphic a distance R(t_{A}) from a point indicative of G(t_{A}). In some embodiments, R(t_{A}) is represented by a distance and at least one indicium is displayed including at least one graphic a vertical distance R(t_{A}) from a point indicative of G(t_{A}). In some embodiments, R(t_{A}) is represented by a distance and at least one indicium is displayed including at least one graphic extending a distance R(t_{A}) from a point indicative of G(t_{A}). In some embodiments, R(t_{A}) is represented by a distance and at least one indicium is displayed including at least one graphic extending a vertical distance R(t_{A}) from a point indicative of G(t_{A}). In some embodiments, R(t_{A}) is represented by a distance and at least one indicium is displayed as a first graphic a distance R(t_{A}) above a point indicative of G(t_{A}) and a second graphic a distance R(t_{A}) below the point indicative of G(t_{A}).

The cone of confidence 804 will continually change as the past glucose concentrations 802A change. However, the cone of confidence 804 provides users with a quick visual aid of a projected range of future glucose concentrations. As shown in FIG. 8B, glucose concentrations 802B appearing within the cone of confidence 804 from the time t₀ to the time t_{B} indicates that the cone of confidence 804 had been accurately projected.

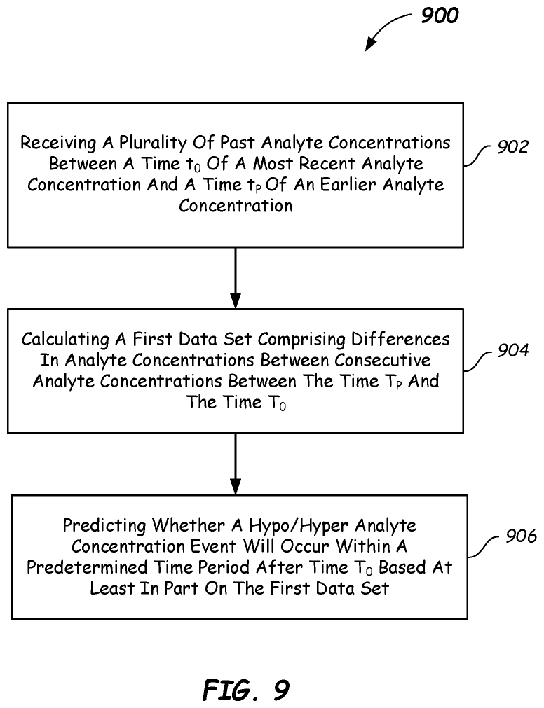

FIG. 9 illustrates a flowchart showing a method 900 of predicting an analyte concentration trend. The method 900 includes, at process block 902, receiving a plurality of past analyte concentrations (e.g., past glucose concentrations 200A, 202A) between a time t₀ of a most recent analyte concentration (e.g., present analyte concentration G(t₀)) and a time t_{P} of an earlier analyte concentration. The method 900 includes, at process block 904, calculating a first data set (e.g., first data set 420A) comprising differences in analyte concentrations between consecutive analyte concentrations between the time t_{P} and the time t₀. The method 900 includes, at process block 906, predicting whether a hypo/hyper analyte concentration event will occur within a predetermined time period (e.g., time period F) after the time t₀ based at least in part on the first data set. For example, assume a present time measured analyte concentration of 170 mg/dL and four previously-measured analyte concentrations of 150, 140, 120, and 105 mg/dL in order of most recent to least recent, measured at three-minute time intervals. The first data set of differences in measured analyte concentrations would be {20,10,20,15} (i.e., 170−150=20, 150−140=10, 140−120=20, and 120−105=15). Based at least in part on this first data set and on, e.g., a large number of analyte concentration measurements of other individuals used to train an artificial intelligence model or the like used in the method, the method may predict a very high likelihood of a hyper-analyte concentration event occurring in the next three minutes.

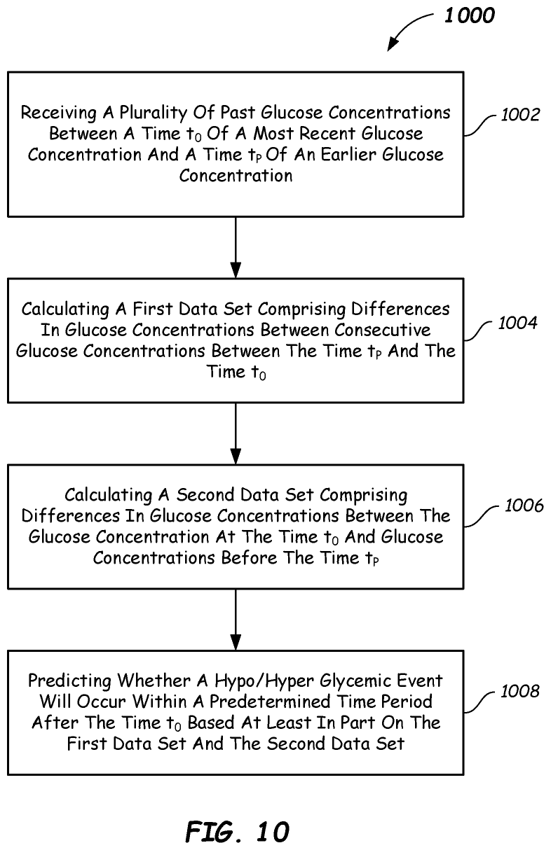

FIG. 10 illustrates a flowchart showing a method 1000 of predicting a glucose concentration trend. The method 1000 includes, at process block 1002, receiving a plurality of past glucose concentrations (e.g., past glucose concentrations 200A, 202A) between a time t₀ of a most recent glucose concentration and a time t_{P} of an earlier glucose concentration. The method 1000 includes, at process block 1004, calculating a first data set (e.g., first data set 420A) comprising differences in glucose concentrations between consecutive glucose concentrations between the time t_{P} and the time t₀. The method 1000 includes, at process block 1006, calculating a second data set (e.g., second data set 402B) comprising differences in glucose concentrations between the glucose concentration at the time t₀ and each glucose concentration before the time t₀. The method 1000 includes, at process block 1008, predicting whether a hypo/hyper glycemic event will occur within a predetermined time period (e.g., time period F) after the time t₀ based at least in part on the first data set and the second data set. For example, assume a present time measured glucose concentration of 105 mg/dL and four previously-measured glucose concentrations of 120, 140, 150 and 160 mg/dL in order of most recent to least recent, measured at five-minute time intervals. The first data set of differences in measured glucose concentrations would be {−15, −20, −10, −10} (i.e., 105−120=−15, 120−140=−20, 140−150=−10, and 150−160=−10). The second data set of differences would be {−15, −35, −45, −55} (i.e., 105−120=−15, 105−140=−35, 105−150=−45, and 105−160=−55). Based at least in part on these first and second data sets and on, e.g., a large number of glucose concentration measurements of other individuals used to train an artificial intelligence model or the like used in the method, the method may predict a high likelihood of a hypoglycemic event occurring in the next ten minutes.

The foregoing description discloses only example embodiments. Modifications of the above-disclosed apparatus and methods which fall within the scope of this disclosure will be readily apparent to those of ordinary skill in the art.

What is claimed is:

1. A method of predicting an analyte concentration trend, comprising:
receiving a plurality of past measured analyte concentrations between a time t₀ of a most recent measured analyte concentration and a time t_{P} of an earlier measured analyte concentration;
calculating a first data set comprising differences in measured analyte concentrations between consecutive measured analyte concentrations between the time t_{P} and the time t₀; and
predicting whether a hypo/hyper analyte concentration event will occur within a predetermined time period after the time t₀ based at least in part on the first data set.

2. The method of claim 1, further comprising calculating a second data set comprising differences in measured analyte concentrations between a measured analyte concentration at the time t₀ and each measured analyte concentration before the time t₀, wherein the predicting comprises predicting whether the hypo/hyper analyte concentration event will occur within the predetermined time period after the time t₀ based at least in part on the first data set and the second data set.

3. The method of claim 2, wherein predicting whether the hypo/hyper analyte concentration event will occur is based solely on the first data set and the second data set.

4. The method of claim 1, wherein the analyte concentration trend is a glucose concentration trend, and the hypo/hyper analyte concentration event is a hypo/hyper glycemic event.

5. The method of claim 1, wherein the predicting whether the hypo/hyper analyte concentration event will occur is performed using an algorithm comprising at least one of artificial intelligence, a machine learning model, and a neural network.

6. The method of claim 1, wherein the predetermined time period ranges from ten minutes to forty-five minutes.

7. The method of claim 1, wherein the predicting whether the hypo/hyper analyte concentration event will occur is performed using an algorithm comprising at least one of: a trained model, a gradient boosted regression tree, and a linear regression.

8. The method of claim 1, wherein the time between to and t_{P} ranges from ten minutes to forty-five minutes.

9. The method of claim 1, wherein the past analyte concentrations between the time t₀ and the time t_{P} are at increments between one minute and five minutes or between two minutes and four minutes.

10. The method of claim 1, further comprising predicting an analyte concentration trend of “rising,” “steady,” “falling,” “up fast,” “up slow,” “down slow,” or “down fast” based at least in part on the first data set.

11. The method of claim 1, wherein the predicting whether the hypo/hyper analyte concentration event will occur is based solely on the first data set.

12. The method of claim 1, further comprising calculating a probability that the predicting whether the hypo/hyper analyte concentration event will occur is correct.

13. The method of claim 1, wherein the predicting whether the hypo/hyper analyte concentration event will occur comprises predicting whether at least one analyte concentration in the predicted analyte concentration trend will exceed a first predetermined analyte concentration or fail to reach a second predetermined analyte concentration within the predetermined time period.

14. The method of claim 13, comprising calculating a probability that the at least one analyte concentration will exceed the first predetermined analyte concentration or fail to reach the second predetermined analyte concentration.

15. The method of claim 1, wherein the predicting at least one analyte concentration comprises predicting a first analyte concentration within the predetermined time period after the time t₀ and a second analyte concentration within a second predetermined time period after the time t₀.

16. The method of claim 15, further comprising calculating a first probability that predicting the first analyte concentration is correct and a second probability that the predicting the second analyte concentration is correct.

17. The method of claim 1, further comprising training a machine learning model to predict whether the hypo/hyper analyte concentration event will occur, wherein training the machine learning model comprises:
performing a plurality of analyte concentration measurements of at least one individual to generate measured analyte concentrations; and
training the machine learning model based on the measured analyte concentrations.

18. A method of predicting a glucose concentration trend, comprising:
receiving a plurality of past measured glucose concentrations between a time t₀ of a most recent measured glucose concentration and a time t_{P} of an earlier measured glucose concentration;
calculating a first data set comprising differences in measured glucose concentrations between consecutive measured glucose concentrations between the time t_{P} and the time t₀;
calculating a second data set comprising differences in measured glucose concentrations between a measured glucose concentration at the time t₀ and each measured glucose concentration before the time t₀; and
predicting whether a hypo/hyper glycemic event will occur within a predetermined time period after the time t₀ based at least in part on the first data set and the second data set.

19. An event detector, comprising:
a processor configured to execute computer-readable instructions that cause the processor to:
receive a plurality of past measured glucose concentrations between a time t₀ of a most recent measured glucose concentration and a time t_{P} of an earlier measured glucose concentration;
calculate a first data set comprising differences in measured glucose concentrations between consecutive measured glucose concentrations between the time t_{P} and the time t₀; and
predict whether a hypo/hyper glycemic event will occur within a predetermined time period after the time t₀ based at least in part on the first data set.

20. The event detector of claim 19, wherein the processor is further configured to execute computer-readable instructions that cause the processor to:
calculate a second data set comprising differences in measured glucose concentrations between a measured glucose concentration at the time t₀ and each measured glucose concentration before the time t₀; and
predict whether the hypo/hyper glycemic event will occur based at least in part on the first data set and the second data set.
